(19) United States
(12) Patent Application Publication
(10) Pub. No.: US 2024/0245459 A1
(43) Pub. Date: Jul. 25, 2024

(54) SYSTEMS AND METHODS FOR SURGICAL THREE-DIMENSIONAL MAPPING AND PATH DETERMINATION

(71) Applicant: HT MEDICAL, LLC, Tulsa, OK (US)

(72) Inventors: Ryan PHILLIPS, Orlando, FL (US); Robert HIRSCHL, Windermere, FL (US)

(73) Assignee: HT MEDICAL, LLC, Tulsa, OK (US)

(21) Appl. No.: 18/418,251

(22) Filed: Jan. 20, 2024

Related U.S. Application Data
(60) Provisional application No. 63/440,253, filed on Jan. 20, 2023.

(51) Int. Cl. A61B 34/10; A61B 17/70

(52) U.S. Cl. CPC A61B 34/10; A61B 17/70; A61B 2034/104; A61B 2034/105; A61B 2034/107

(57) ABSTRACT

A method for spinal surgery may include receiving a planned rod contour and using a flexible surgical sensor to generate a three-dimensional path, wherein the three-dimensional path may represent a spinal contour of a patient. The method may further include bending a spinal rod according to one of: the planned rod contour, the spinal contour of a patient, and a modified contour derived from the planned rod contour and/or the spinal contour of the patient.

### CROSS-REFERENCE TO RELATED APPLICATIONS

The application claims the benefit of U.S. Provisional Patent Application Ser. No. 63/440,253 filed Jan. 20, 2023 and entitled SYSTEMS AND METHODS FOR SURGICAL THREE-DIMENSIONAL MAPPING AND PATH DETERMINATION, which is incorporated by reference as though set forth herein in its entirety.

### TECHNICAL FIELD

The present disclosure relates to surgical devices, systems, and methods. More specifically, the present disclosure relates to devices, systems, and methods for computer aided design and manufacture of medical implants directed for spinal surgery.

### BACKGROUND

Spinal fixation procedures utilizing pedicle screws and rod-based fixation assemblies can be used to correct spinal conditions such as degenerative disc disease, kyphosis of the spine, spondylolisthesis, spinal deformities, or other spinal conditions through minimally invasive or invasive spinal surgery. For example, two or more bone anchor assemblies may be secured into bone structures of a patient's vertebrae with surgical implements (e.g., connecting rods or cords) secured between adjacent bone anchor assemblies in order to stabilize one or more vertebral joints of a patient. These surgical implements typically run longitudinally along the length of the patient's spine between or along adjacent bone anchor assemblies. However, these surgical implements can be arranged in a variety of positions and/or configurations (e.g., including the use of multiple connecting rods, cords, and/or cross-bars, where desired) in view of a patient's specific anatomy and/or a specific spinal correction.

Unfortunately, the process of determining the shape, length, and geometry of the surgical implements can be difficult when the shaping is performed manually or when imaging alone is used to determine spinal position, deformations, or alignment.

Other surgical procedures, such as endoscopic procedures, hemivertebral excision, vertebral column resection (VCR), VEPTR, VBT, video-assisted thoracoscopic surgery—i.e., using an anterior approach (VAPTS), or determining orthopedic biomechanics, utilize additional surgical implements (e.g., endoscope, laparoscope, stents, etc.), requiring either multiple and/or increasingly precise insertions into a patient. Unfortunately, similar to spinal surgeries, relying on imaging alone for positioning or aligning surgical implements may complicate, increase risk, or otherwise reduce the efficacy of surgical procedures that use these surgical implements.

Accordingly, improved surgical systems, methods, instruments, and devices that simplify, enhance, or improve these processes, uses, and systems would be desirable.

### SUMMARY

The various systems and methods of the present disclosure have been developed in response to the present state of the art, and in particular, in response to the problems and needs in the art that have not yet been fully solved by currently available devices and methods for surgical three-dimensional mapping and path determination.

In some embodiments, a method for spinal surgery may include receiving a planned rod contour and using a flexible surgical sensor to generate a three-dimensional path, wherein the three-dimensional path may represent a spinal contour of a patient. The method may further include bending a spinal rod according to one of: the planned rod contour, the spinal contour of a patient, and a modified contour derived from the planned rod contour and/or the spinal contour of the patient.

In the method of any preceding paragraph, the method may further include preoperatively generating the planned rod contour based on a desired spinal contour of the patient.

In the method of any preceding paragraph, the spinal contour of the patient may be a current spinal contour of the patient.

In the method of any preceding paragraph, the spinal contour of the patient may be a desired spinal contour of the patient.

In the method of any preceding paragraph, the flexible surgical sensor may be received by one or more pedicle screws to facilitate conformity of the flexible surgical sensor with the spinal contour.

In the method of any preceding paragraph, the spinal rod may include one or more notches configured to facilitate separation of a bent portion of the spinal rod from an unbent portion of the spinal rod.

In the method of any preceding paragraph, the planned rod contour may be divided into multiple spinal levels, wherein modifications may be selected independently for each spinal level.

In the method of any preceding paragraph, the method may further include adjusting the planned rod contour based on the spinal contour of the patient to generate the modified contour.

In the method of any preceding paragraph, the method may further include calculating a delta vector based on the spinal contour of the patient and the planned rod contour; and generating the modified contour based on the delta vector.

In some embodiments, a method for spinal surgery may include receiving a planned rod contour and using a flexible surgical sensor to generate a three-dimensional path, wherein the three-dimensional path may represent a spinal contour of a patient. The method may further include adjusting the planned rod contour based on the spinal contour of the patient to generate a modified contour.

In the method of any preceding paragraph, the method may further include bending a spinal rod according to the modified contour.

In the method of any preceding paragraph, the flexible surgical sensor may be received by one or more pedicle screws to facilitate conformity of the flexible surgical sensor with the spinal contour.

In some embodiments, a system for facilitating spinal surgery may include: a non-transitory computer-readable storage medium configured to store a planned rod contour and a flexible surgical sensor configured to measure a spinal contour of a patient. The system may further include a display device configured to overlay the planned rod contour with the spinal contour.

In the system of any preceding paragraph, the system may also include a rod bender configured to bend a spinal rod according to the planned rod contour.

In the system of any preceding paragraph, the spinal contour of the patient may be a current spinal contour of the patient.

In the system of any preceding paragraph, the spinal contour of the patient may be a desired spinal contour of the patient.

In the system of any preceding paragraph, the storage medium may be further configured to store data from the flexible surgical sensor.

In the system of any preceding paragraph, the flexible surgical sensor may be configured to be received by one or more pedicle screws to facilitate conformity of the flexible surgical sensor with the spinal contour.

In the system of any preceding paragraph, the system may also include a processor configured to receive adjustment parameters from a user, and based on the adjustment parameters, generate a modified contour.

In the system of any preceding paragraph, the system may also include a processor configured to receive user modifications to the planned rod contour, and to modify the planned rod contour per the user modifications to generate a modified contour.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings in which:

FIG. 1 is a flowchart of a method 100 for three-dimensional (3D) mapping of a surgical site of a patient, according to an embodiment of the present disclosure;

FIGS. 2A to 2C are views of a notched surgical implement, according to an embodiment of the present disclosure;

FIG. 3 is a perspective view of a system for aligning a surgical implement relative to a three-dimensionally mapped surgical site, according to an embodiment of the present disclosure;

FIG. 4 is a perspective view of the surgical site and flexible surgical sensor of FIGS. 2A to 2C;

FIG. 5 is a top perspective view of the surgical site and flexible surgical sensor of FIGS. 2A to 2C;

FIG. 6 is a perspective view of the rod bender of FIGS. 2A to 2C;

FIG. 7 is a perspective side view of the rod bender and patient of FIGS. 2A to 2C;

FIG. 8 is a top perspective view of another embodiment of a system for aligning a surgical implement relative to a three-dimensionally mapped surgical site; and

FIGS. 9A to 9C are views of passive surgical sensor systems, according to embodiments of the present disclosure.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the invention, as claimed, but is merely representative of exemplary embodiments of the technology.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. These descriptive terms may be applied to an animate or inanimate body.

The improvements discussed herein may generally be utilized in medical investigations, diagnostic procedures, and/or treatments.

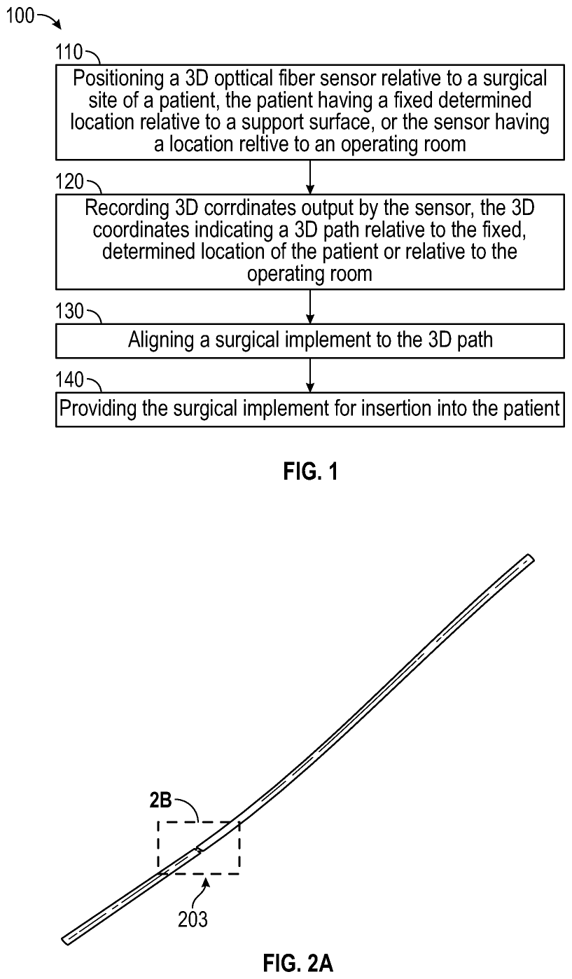

FIG. 1 illustrates a flowchart of a method 100 for three-dimensional mapping of a surgical site of a patient. In general, the method 100 may include use of an active three-dimensional (3D), flexible surgical sensor. The flexible surgical sensor may include any flexible positional sensing mechanism. The flexible surgical sensor may have a flexible member that can detect the three-dimensional position of various segments along its length, either directly (for example, via an array of position sensors) or via detection of one or more other properties such as deflection in the flexible member. In some embodiments, the flexible surgical sensor may include optical fiber. More specifically, in some embodiments, the flexible surgical sensor may be a fiber optic shape sensing device, as marketed by The Shape Sensing Company of Austin, Texas and Santa Barbara, California.

Additional sensors may be used instead of or in combination with the fiber optic shape sensing device. For example, one or more (e.g., array) of Fiber Bragg Grating (FBG) sensors may be used. In these embodiments, the surgical implement may be made of, or include as a component of its material composition, fiber reinforced thermoplastic composites, photonic crystal fibers (PCF), hydrogen-loaded PCF, or combinations thereof. The FGB sensors may be configured as refractive sensors using the guided light within an optical fiber.

The flexible surgical sensor may be housed within an elongate tube. The tube and flexible surgical sensor may have an inferior end and a superior end. The method 100 may include use of a memory and a display device that are coupled to (e.g., operably connected to) a controller including a processor. The method 100 may also utilize an automatic (i.e., non-manual) rod bending machine (i.e., rod bender) comprising bend script derived from 3D coordinates received from the controller and flexible surgical sensor.

In preparation for the method 100, one or more initial procedures may occur. For example, an incision may be made at a surgical site for inserting the superior end of the flexible surgical sensor at the surgical site. By way of another example, straps, table surfaces, surgery staff, or other devices and means may be used to position the patient such that the spine is in an ideal or substantially ideal position and alignment based on the age, weight, and size of the patient. After the incision has been made, the method 100 may begin with a step 110 in which the 3D flexible surgical sensor is positioned relative to the surgical site of the patient. By way of yet another example, the method 100 may be preceded by a calibration of the flexible surgical sensor. The calibration may be automatic, semi-automatic, or manual. For example, after the sensor is connected to a rod bender, LED indicators and firmware pre-programmed on the rod bending machine may automatically indicate whether or not the sensor is calibrated correctly (e.g., red for not calibrated correctly, and green for calibrated correctly). In other embodiments, a display device may be a component of the system configured to perform method 100, such that the calibration may be semi-automatic, or fully manual based on inputs detected and displayed on the display device.

The positioning of the 3D flexible surgical sensor may be internal relative to the body of the patient, such as by inserting the sensor through the incision and connecting or contacting multiple implanted pedicle screws of the patient. For example, the superior end of the 3D flexible surgical sensor may be in contact with or temporarily coupled to one or more superior implanted pedicle screws (e.g., using rod crimps, posts, hooks, threads, or combinations thereof) of the patient, while an inferior end of the 3D flexible surgical sensor may be in contact with or temporarily coupled to one or more inferior implanted pedicle screws (e.g., or iliac screws) of the patient. If there are implanted pedicle screws between the inferior and superior pedicle screws, the flexible surgical sensor may be contacting or temporarily coupled to those screws as well. The flexible surgical sensor may be configured to be received by one or more pedicle screws implanted in the patient to facilitate conformity of the flexible surgical sensor with the spinal contour. In other embodiments, the positioning of the 3D flexible surgical sensor may be external or on top of skin (e.g., along an outer surface of a spine and skin of the patient).

In at least one embodiment, the patient may have a fixed, determined location relative to a support surface, such as an operating table or a floor of the operating room. For example, grid lines, indicators, markers, or one or more locating sensors may determine and ensure accurate, fixed, repeated identical locations (e.g., or substantially identical locations) of the patient such that the 3D coordinate system of the flexible surgical sensor is the same, or is calibrated to be the same, before each iterative 3D mapping or surgical use. In other embodiments, the 3D output of the flexible surgical sensor may be dependent on the patient being within the operating room, and independent of the precise location of the patient within the operating room. For example, the 3D flexible surgical sensor outputs 3D coordinates relative to the operating room where the patient is located, such that as long as the 3D flexible surgical sensor is positioned at the surgical site of the patient (e.g., along the spine), the 3D coordinates output by the sensor are provided relative to a determined coordinate (e.g., absolute location within the operating room) of one end (e.g., superior or inferior end of the sensor) and multiple preceding or proceeding 3D coordinates or points relative to the first coordinate and along the sensor. Grid lines, indicators, markers, or a locating sensor of the operating room where the patient is located may be used to ensure the 3D coordinates output by the sensor are always locationally incremented or regressed identically (e.g., incrementations or regressions relative to fixed points in the operating room) over time (e.g., such as for subsequent evaluations to determine the status/alignment of the spine). In other words, as long as the patient is located within the operating room, the 3D output of the sensor is provided and interpreted relative to iterative or regressive data points of the sensor and the position of the sensor within the operating room (i.e., without regard to an exact location of the patient within the operating room).

In some embodiments, the absolute position of each segment of the flexible surgical sensor need not be obtained. Rather, it may be sufficient to obtain the position of each segment relative to the segment(s) adjacent to it. For example, the inferior end of the flexible surgical sensor may be designated as a base segment. The position of each other segment may be provided by the flexible surgical sensor, relative to the base segment. This relative position may be sufficient to obtain the shape data needed to bend a surgical implement, such as a spinal rod, based on the disposition of the flexible surgical sensor.

Once the flexible surgical sensor is positioned relative to the surgical site, the method 100 may proceed to step 120 in which the display device, the memory, and/or the controller may be used to record 3D coordinates that are output by the sensor. The 3D coordinates from the sensor indicate a 3D path, thereby determining or mapping a curvature of the spine.

Once the 3D coordinates are output from the flexible surgical sensor and recorded using the display device, the memory, and/or the controller, the method 100 may proceed to a step 130 in which a surgical implement is aligned to the 3D path determined by the coordinates output by the sensor. For example, if the patient is positioned and held in place such that the spinal column is appropriately aligned, the alignment of the surgical implement (e.g., scoliosis rod, Harrington rod, magnetically controlled growing rod, etc.) may be substantially equivalent to the 3D coordinates output by the sensor at the time it is initially inserted or positioned relative to the surgical site. In other embodiments, the 3D coordinates output by the sensor may indicate an unaligned or deformed spine (e.g., such as with scoliosis), and the alignment of the surgical implement (e.g., scoliosis rod) may be based on a progressive, iterative alignment of the spine over time or tightening of pedicle screws, meaning that the alignment of the surgical implement is according to a future or intended alignment relative to the surgical site (e.g., spine) of the patient. In yet another embodiment, a surgical implement, such as an endoscope or a laparoscope, may be aligned with the exact, complex, tortuous 3D path of the flexible surgical sensor, based on the 3D coordinates and path output by the sensor, thereby minimizing or eliminating additional tissue damage caused by subsequent insertions of either the flexible surgical sensor or one or more surgical implements.

In at least one embodiment, a delta vector (e.g., translation vector) is calculated, indicating a positional and directional difference between the 3D coordinates/path measured by the flexible surgical sensor and the absolute, real-time position, or shape of the surgical implement. For example, a second flexible surgical sensor (e.g., positioned within or along the surgical implement) or a second reading/recording of the first flexible surgical sensor positioned along the surgical implement may be compared to the initial reading/recording of the flexible surgical sensor positioned within the patient. Additionally, or alternatively, a reading of the current spinal contour using the flexible surgical sensor may be compared to the shape of an existing surgical implement and/or a planned rod contour. The comparison generates a delta vector. The delta vector may indicate how the surgical implement should be manipulated (e.g., bent) to produce alignment with either the initial 3D coordinates of the sensor (i.e., 3D path) or the future/intended coordinates of the surgical site (e.g., spine). A modified contour may be generated based on the delta vector. In at least one embodiment, the future/intended coordinates indicate a properly aligned spine based on factors such as the patient's age, weight, and size. For example, the 3D path may be used by the rod bending machine to determine where one or more bends (e.g., direction, shape, convex length/depth, concave length/depth, etc.) should be made in the rod. In other embodiments, a cord may be used instead of or in combination with rods, where the cords are aligned to the 3D path of step 130 by tightening pedicle screws in which the cord is inserted, thereby straightening or properly aligning the spine. In at least one embodiment, calculating the delta vector may include one or more of 3D translation, rotation, scaling, reflection, or shearing, Mathematically the delta vector, and a translation thereof, may be computed using one or more matrixes (e.g., 3D translation matrix).

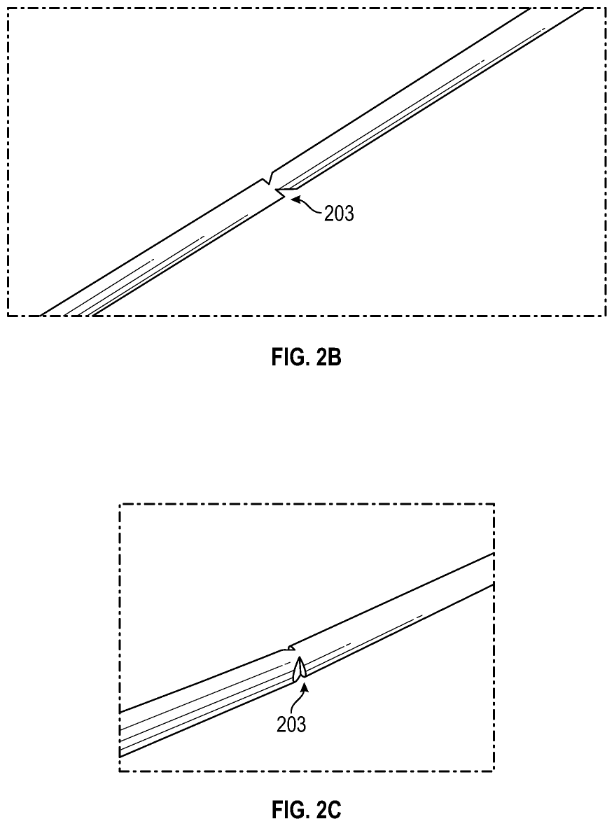

Once the surgical implement is aligned to the 3D path of the flexible surgical sensor, the method 100 may proceed to a step 140 in which the surgical implement is provided for insertion into the patient. For example, the automatic (non-manual) rod bender may cut the aligned rod from a remaining supply, or rod feed, that is connected to the rod bender. For example, the straight or unbent portion of the rod may be inserted into or remain within the rod bender, such that the rod bender cuts or snips the rod, separating the bent portion from the straight portion. Referring to FIGS. 2A to 2C, in other embodiments, varying lengths of rod may be used to form bent rods, where a length of rod includes one or more notches 203 at a specific distance (e.g., 100, 110, 120, 130, 140, or 150 mm) from an end (e.g., superior end) of the rod. After the rod bender bends or shapes the rod, a user or the rod bender itself, may flex the rod at the junction of a notch 203 to snap the bent portion, separating it from the straight, unbent portion.

In some embodiments, the alignment of the surgical implement to the 3D path of the flexible surgical sensor may occur at a different facility and/or on at different time or day than the obtaining of the 3D path. Thus, providing the surgical implement for insertion into the patient may include a shipment and receipt of the surgical implement (e.g., rod), after which a surgeon may perform the insertion of the surgical implement into the patient at the surgical site. In at least one embodiment, an endoscope or a laparoscope may comprise the surgical implement, such that providing the surgical implement for insertion into the patient further includes maintaining the alignment of the surgical implement to the 3D path determined by the active flexible surgical sensor, where the maintaining of the alignment occurs after the insertion of the surgical implement into the patient and throughout the surgical operation until completion.

Additionally, or alternatively, the bending of the surgical implement may be based on preoperative surgical planning and may occur at a different facility and/or on at different time or day than the surgical planning. In an embodiment, the surgical implement may be bent according to a planned rod contour that may be based on preoperative surgical planning. Alternately, the planned rod contour may be based on interoperative surgical planning. After receiving the surgical implement, the flexible surgical sensor may be used to determine the 3D path of the surgical implement.

Referring again to FIG. 1, once providing, inserting, and/or maintaining alignment of the surgical implement in step 140 occurs, the method 100 may end. Alternatively, or in addition thereto, the method 100 may proceed to (i.e., repeat) any or all of steps 110-140, as will be discussed below.

Once the surgical implement has been inserted into and positioned at the surgical site of the patient, the method 100 may repeat step 110 in which the flexible surgical sensor may be repositioned relative to the surgical site of the patient. This repositioning may be to ascertain the accuracy of the positioning that already occurred, or in at least one embodiment, this repositioning may be for the purposes of placing second, third, or more additional rods, cords, or cross-bars within the patient. For example, the planned rod contour may be divided into multiple spinal levels, so that modifications may be selected independently for each spinal level.

Once the flexible surgical sensor has been re-inserted or re-positioned at the surgical site of the patient, the method 100 may repeat step 120 in which the 3D coordinates output by the sensor may be recorded to determine or indicate a second 3D path of the flexible surgical sensor. The second 3D path may represent the current spinal contour of the patient. Additionally, or alternatively, the second 3D path may represent a desired spinal contour of the patient.

Once the 3D coordinates are again recorded, the method 100 may repeat step 130 in which additional alignments or adjustments are made to the surgical implement at the surgical site of the patient to further align the surgical implement to the second 3D path of the flexible surgical sensor. In other embodiments, the step 130 may include aligning second, third, or more rods/cords to the second 3D path determined by the flexible surgical sensor.

Once the first, second, and/or third surgical implement is determined to be in proper alignment with the first, second and/or third 3D path, the method 100 may proceed to repeating step 140 in which a second and/or third surgical implement (e.g., second or third scoliosis rod or cord) is provided for insertion into the patient. Once all of the surgical implements determined to be necessary for the procedure have been provided, inserted, and/or aligned within the patient, the method 100 may end.

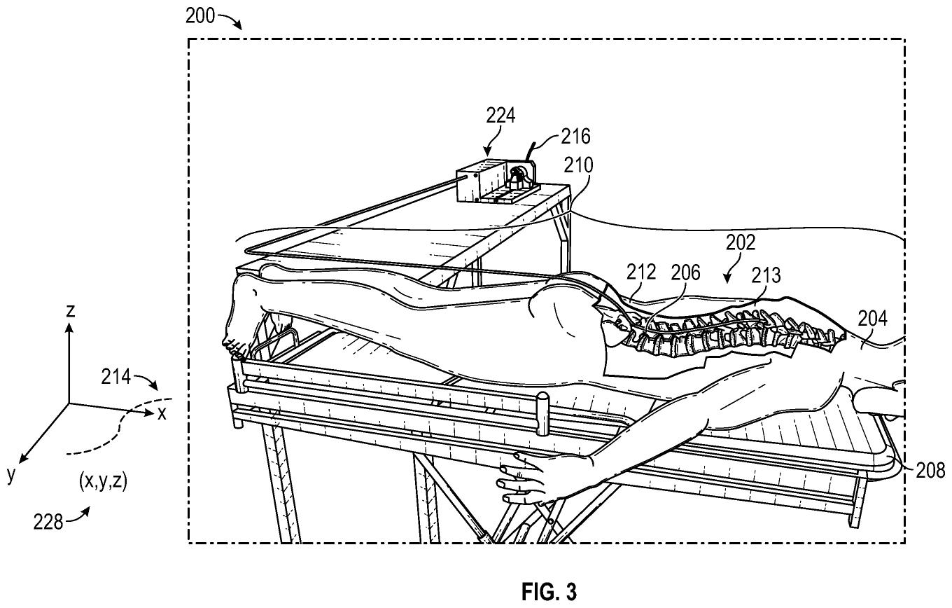

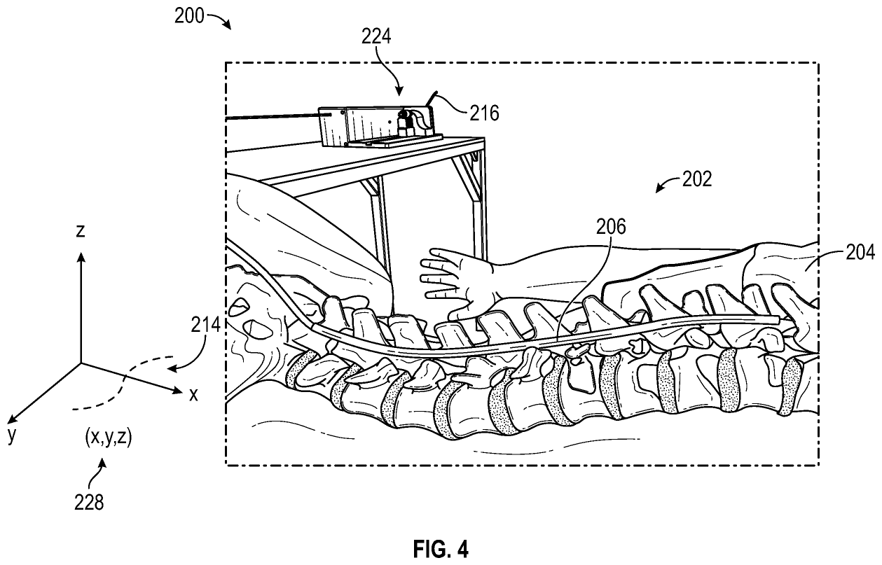

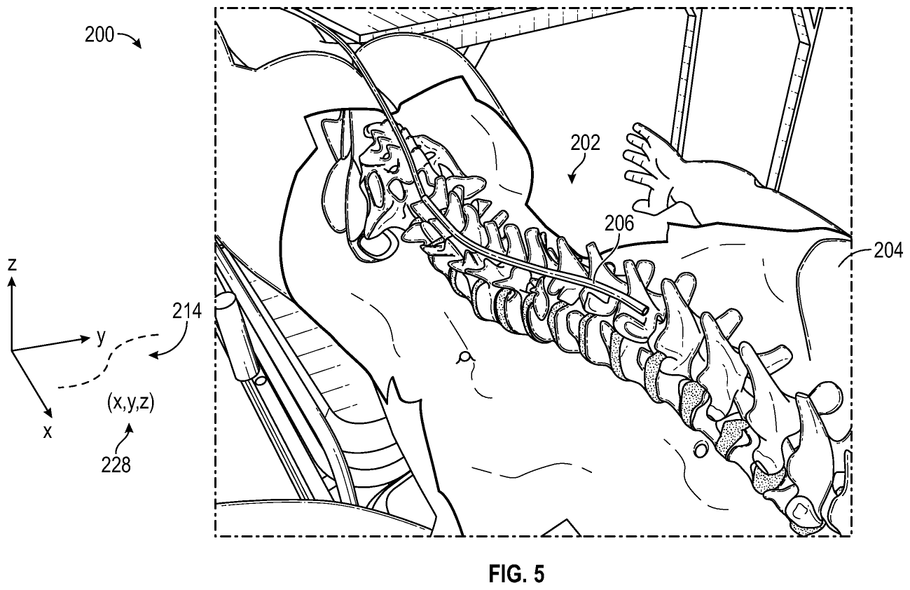

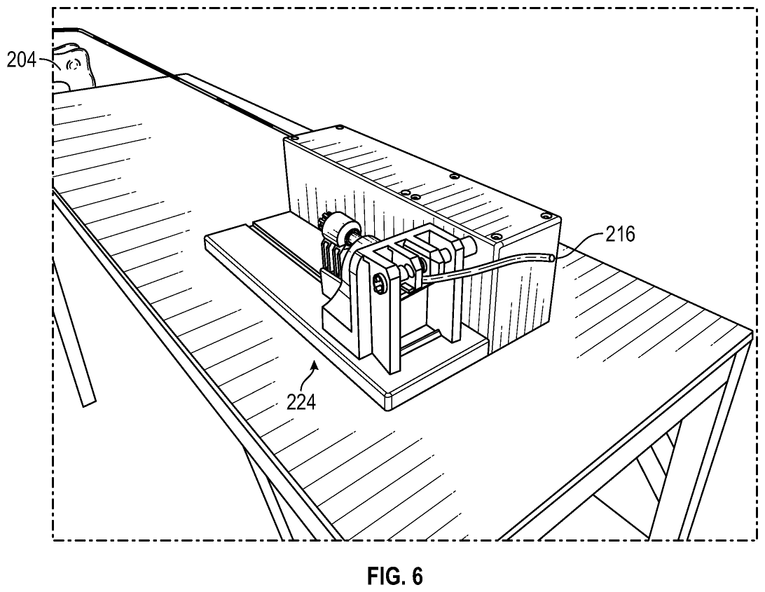

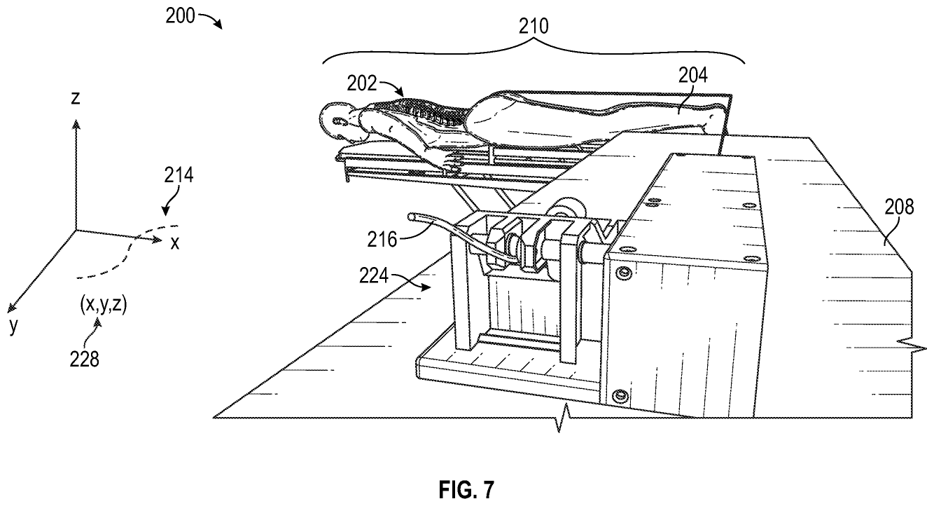

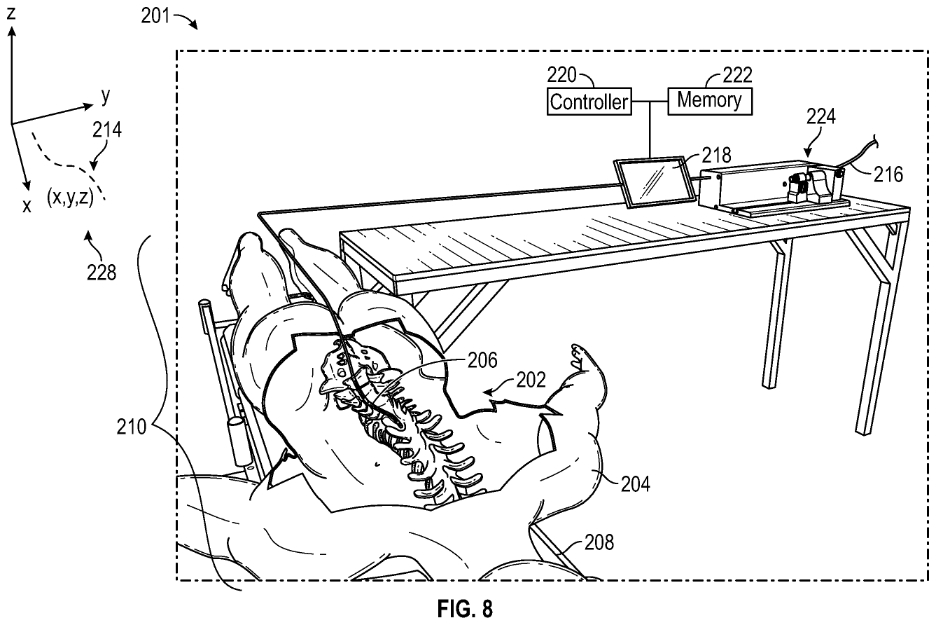

Referring to FIG. 3, a system 200 for aligning a surgical implement relative to a three-dimensionally mapped surgical site is shown. FIG. 3 shows a perspective view, FIG. 4 shows an exploded side-perspective view of the surgical site. FIG. 5 shows a top perspective view of the surgical site. FIG. 6 shows a perspective view of the rod bender. FIG. 7 shows a side perspective view of the patient and the rod bender of the system. FIG. 8 shows a perspective view of another embodiment of a system for aligning a surgical implement relative to a three-dimensionally mapped surgical site.

As shown, the system 200 may be used at a surgical site 202 of a patient 204, where an active flexible surgical sensor 206 may be inserted, positioned, and/or aligned within the patient 204. The flexible surgical sensor 206 may be immune to electromagnetic interference and changes in temperature. A support surface 208, such as an operating table, may be used for supporting the patient during the surgery, providing alignment to the spine of the patient 204 prior to and during the surgery, or obtaining and maintaining a fixed, determined location 210 of the patient 204 during the first surgery and during subsequent examinations and/or surgeries. The flexible surgical sensor 206 has an inferior end 212 and a superior end 213, where at least the superior end 213 is shaped and configured for insertion (e.g., substantially parallel to a superior-inferior or cephalad-caudal axis) at the surgical site 202. For example, the superior end 213 may be domed, include a camera or a sensor, or include other apparatuses for performing surgery, such as cutters or clamps.

In at least one embodiment, a 3D path 214 generated by the flexible surgical sensor 206 may be used to determine a shape automatically provided (e.g., via bending) to the surgical implement 216 (e.g., rod) by the rod bender 224. The 3D path is based on the 3D coordinates of the flexible surgical sensor 206 and is used to generate bend script for the rod bender 224. The 3D path may be determined relative to a 3D coordinate system 228 utilized by the flexible surgical sensor 206 and/or utilized by a locating sensor within the operating room. At least upon initial installation and setup, the flexible surgical sensor 206 may be calibrated to the 3D coordinate system 228. In other embodiments, the flexible surgical sensor 206 may be recalibrated according to a schedule or a set duration, such as daily, weekly, monthly, semi-annually, annually, etc. According to alternative embodiments, the flexible surgical sensor 206 may not require calibration.

In at least one embodiment, the support surface 208 may include indicators, grid lines, markers, and/or a locating sensor (not shown) for a fixed, determined location 210 of the patient 204 relative to the support surface 208. In other embodiments, the operating room in which the support surface 208 is located may include the indicators, grid lines, markers, or the locating sensor. Such indicators, grid lines, markers, etc. are optional, as in some embodiments, the flexible surgical sensor 206 may only measure the position of its segments relative to each other, rather than to the support surface 208, the operating room, or any other external reference.

Referring to FIG. 8, a perspective view of another embodiment of a system 201 for aligning an active flexible surgical sensor relative to a three-dimensionally mapped surgical site of a patient is shown. The system 201 may be substantially similar to the system 200 except that system 201 includes a display device 218, which may be operated via a graphical user interface and/or any other user interface known in the art. The display device 218 may be configured at least for calibrating the sensor and the 3D coordinate system 228 of the flexible surgical sensor 206 relative to the support surface 208, relative to the patient 204 positioned on the support surface 208, relative to the operating room, or combinations thereof. The display device 218 may be coupled to (e.g., operably connected to) a controller 220 and a memory 222. The memory 222 may serve as a non-transitory storage medium that stores data pertinent to operation of the system. The display device 218 may be configured for displaying the 3D path 214 on a display for the user to visualize the 3D path of the flexible surgical sensor 206. In other embodiments, the display device 218 may be coupled with one or more locating sensors (e.g., camera, 3D camera, dual or triple cameras configured to remove parallax, etc.) in the operating room. In at least one embodiment, the display device 218 may be configured to receive input from the locating sensor(s) to display an augmented reality view of the patient having the 3D path superimposed relative to the patient 204. The display device 218 may be configured to display an overlay of one or more of: a 3D path with a planned rod contour; a planned rod contour with a current spinal contour; a 3D path with a current spinal contour; a first 3D path with a second 3D path; an existing surgical implement with a current spinal contour; an existing surgical implement with a 3D path; and an existing surgical implement with a planned rod contour.

The controller 220 may have a processor operably connected to the display device 218, the flexible surgical sensor 206, and the memory 222 for performing one or more steps or computerized instructions. For example, the controller 220 and processor may be programmed for calibrating the flexible surgical sensor 206 and/or recording three-dimensional coordinates output by the flexible surgical sensor 206. The three-dimensional coordinates may be input by the controller 220 or received by the controller 220 from the flexible surgical sensor 206 to determine and indicate (e.g., display) the three-dimensional path 214 relative to the fixed, determined location 210 of the patient 204 or relative to the operating room. The surgical implement 216 of system 201 may be aligned according to the three-dimensional path 214 for insertion into the patient 204. The processor may be configured to receive adjustment parameters from a user, and based on the adjustment parameters, generate a modified contour. Additionally, or alternatively, the processor may be configured to receive user modifications to the planned rod contour, and to modify the planned rod contour per the user modifications to generate a modified contour.

In an embodiment, the controller 220 and/or the display device 218 may further be operably connected to a non-transitory computer-readable storage medium configured to store data from the flexible surgical sensor and/or a passive surgical sensor system. The data may include, but is not limited to a 3D path and/or a planned rod contour.

One or more surfaces of the surgical implement 216 (e.g., scoliosis rod) and/or pedicle screws may be coated and/or infused (e.g., where, in at least one embodiment, the rod has a porous surface) with an osteogenic substance designed to promote bone growth. For example, various calcium phosphates may be used, including hydroxyapatite (“HA”). Such materials may be provided as a surface layer or coating, or may be seated deeper in a porous structure. The osteogenic coating or infusion may facilitate and/or enhance the osseointegration process during the early stages of healing.

In some embodiments, where the osteogenic material is applied as a coating, the coating may be applied to the entire exterior of the surgical implement 216 and/or pedicle screws. In the alternative, such a coating may be applied only to a porous layer of the surgical implement 216 and/or screws. The thickness of the coating may be within the range of 0.001 μm to 1 μm in thickness, or more precisely, 0.01 μm to 0.1 μm, or yet more precisely, from 0.015 μm to 0.05 μm. In some embodiments, the thickness of the coating may be about 0.02 μm (20 nm). Use of such a thin coating may help to preserve the porosity of the porous layer (or in alternative embodiments, the porosity of the entire implant), while still providing the osteogenic properties mentioned above. The thin coating may additionally or alternatively eliminate at least some of the risks associated with thicker osteogenic coatings, such as poor coating integration and poor mechanical stability.

In addition to or in the alternative to a coating, the osteogenic material may be incorporated into the material of the rod. For example, the surgical implement 216 may include a sheath, a tubing, or an exterior that is made of HA PEEK, or a combination of HA PEEK and porous PEEK and/or porous HA PEEK.

Additionally, or alternatively, the osteogenic material may be infused into the material of which the surgical implement 216 is formed, or the porous exterior portion thereof. For example, the exterior layer of the surgical implement 216 may be formed of porous PEEK infused with HA. The HA may be distributed throughout the porous PEEK material, making it a porous PEEK HA structure. In other words, the surgical implement 216 may be formed of a combination of solid and porous PEEK materials, with the PEEK existing as a layer such as porous layer, having HA infused only throughout the porous layer.

The solid, rod-like shape of the surgical implement 216, as illustrated, is merely exemplary. Surgical implements 216 according to the present disclosure may be made in a wide variety of shapes and sizes. For example, smooth, hollow tube-like, elongate structures, or combinations thereof, may be used. Additionally, ridges, grooves, textures, or other shapes, geometries, features, or combinations thereof, may be used in the formation of the surgical implement for structural support, bone growth/fusion enhancement, or for other purposes described herein. In at least one embodiment, a hollow rod may be used to receive the flexible surgical sensor 206 or a second flexible surgical sensor to be used as a template, indicating in real-time the bends and deformations applied to the rod, which may increase the accuracy of the alignment of the bends to the first 3D mapping of the surgical site 202 of the patient 204.

In some embodiments, the surgical implement 216 may not have a rodlike shape at all. For example, the surgical implement 216 may be a patient-specific cutting guide. The flexible surgical sensor 206 may be placed on the surface of a bone proximate a resection site. Its 3D shape may be used to design and/or fabricate a cutting guide with a patient-specific surface configured to fit on the bone to ensure stable, optimal positioning of the cutting guide during the resection procedure.

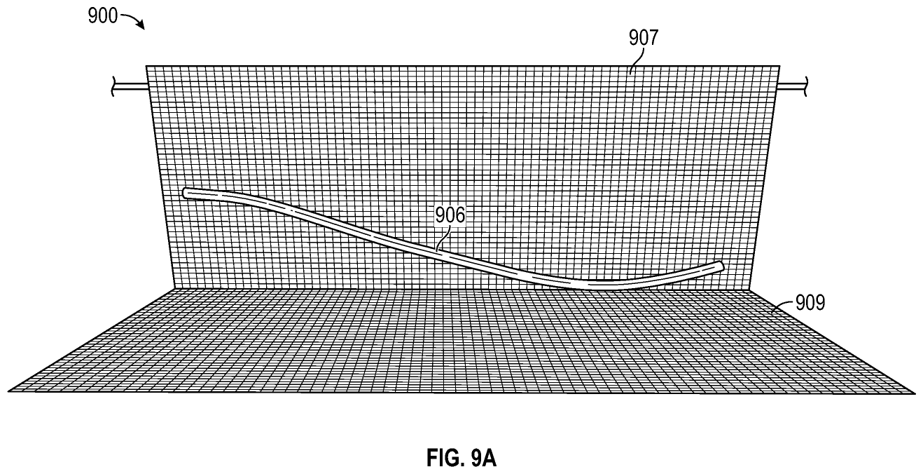

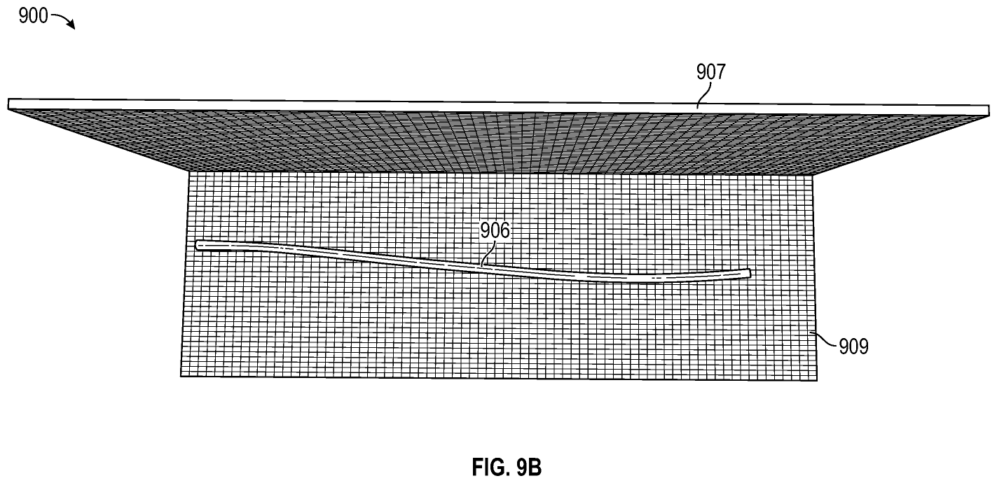

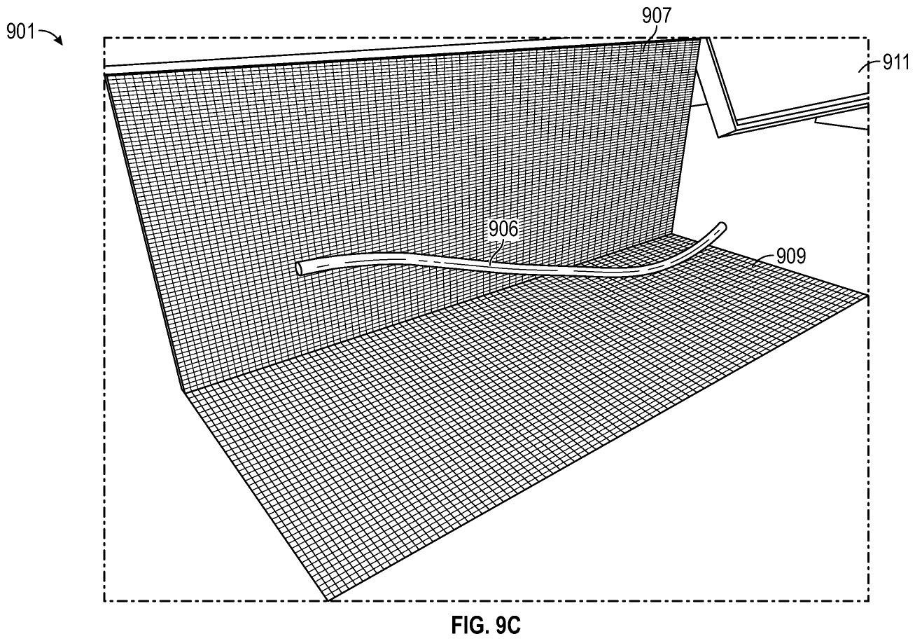

Referring to FIGS. 9A and 9B, in some embodiments the system 200 and/or the system 201 may use—in conjunction with or separate from the active flexible surgical sensor 206 and/or FBGs—a passive surgical sensor system 900. The passive surgical sensor system 900 may include a passive surgical sensor 906, which does not include any active sensor components but instead relies on, for example, the properties of the material composition of the passive surgical sensor 906 as detected by a first sensor plane/field 907 and/or a second sensor plane/field 909.

It is important to note that although two planes/fields 907 and 909 are depicted, additional planes may be used. Additionally, although no patient is depicted in FIGS. 9A and 9B, in some embodiments these planes may correspond to the transverse plane, mid-sagittal, and/or parasagittal planes. The planes/fields 907 and 909 may be measured, gridded, or otherwise incremented (e.g., mapped with a coordinate system) for determining spatial relationships of the passive surgical sensor 906 relative to the planes/fields 907 and/or 909.

The planes/fields 907 and 909 may emanate or originate from an imaging device (e.g., camera, sensor, X-ray generator, magnetic resonance imaging (MRI) scanner, CT scanner, or combination thereof). The planes/fields 907 and/or 909 may be used to generate a 3D path (e.g., 3D path 214) for determining the shape/direction of a surgical implement. In other embodiments, the planes/fields 907 and/or 909 may be used to determine a delta vector for manipulations, alterations, or adjustments to an implanted surgical implement.

Referring to FIG. 9C, a passive sensor system 901 may be used in conjunction with or separate from the passive surgical sensor system 900. The passive sensor system 901 is substantially similar to the passive surgical sensor system 900 except that the passive sensor system 901 includes a laser device 911 (e.g., Photoacoustic Topography Through an Ergodic Relay (PATER) device). The laser device 911 replaces or can be used in combination with the imaging device of the passive surgical sensor system 900.

A processor may combine multiple images, such as those resulting from an imaging device or laser device responsible for plane/field 907 and/or 909, into a single multi-dimensional image. The single multi-dimensional image may be used for generating the 3D path and/or determining the delta vector.

It is important to note that although the methods and systems may be described relative to rods, spinal surgery, or a rod bending machine that makes bends based on the 3D path derived from the 3D coordinates of the flexible surgical sensor, the present disclosure encompasses other uses and systems. For example, rather than outputting script as bends by the rod bending machine, the script may instead be used to display an intended motion or how an endoscope or a laparoscope should be subsequently moved to maintain alignment with an original 3D path, or in the generation of a patient-specific cutting guide, as noted above. Many other custom-designed and/or fabricated surgical implements would be envisioned by a person of skill in the art with the aid of the present disclosure.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

1. A method for spinal surgery comprising:
receiving a planned rod contour;
using a flexible surgical sensor to generate a three-dimensional path, wherein the three-dimensional path represents a spinal contour of a patient; and
bending a spinal rod according to one of: the planned rod contour, the spinal contour of the patient, and a modified contour derived from the planned rod contour and/or the spinal contour of the patient.

2. The method of claim 1, wherein the method further comprises preoperatively generating the planned rod contour based on a desired spinal contour of the patient.

3. The method of claim 1, wherein the spinal contour of the patient is a current spinal contour of the patient.

4. The method of claim 1, wherein the spinal contour of the patient is a desired spinal contour of the patient.

5. The method of claim 1, wherein the flexible surgical sensor is received by one or more pedicle screws to facilitate conformity of the flexible surgical sensor with the spinal contour.

6. The method of claim 1, wherein the spinal rod comprises one or more notches configured to facilitate separation of a bent portion of the spinal rod from an unbent portion of the spinal rod.

7. The method of claim 1, wherein the planned rod contour is divided into multiple spinal levels, wherein modifications are selected independently for each spinal level.

8. The method of claim 1, further comprising adjusting the planned rod contour based on the spinal contour of the patient to generate the modified contour.

9. The method of claim 1, further comprising:
calculating a delta vector based on the spinal contour of the patient and the planned rod contour; and
generating the modified contour based on the delta vector.

10. A method for spinal surgery comprising:
receiving a planned rod contour;
using a flexible surgical sensor to generate a three-dimensional path, wherein the three-dimensional path represents a spinal contour of a patient; and
adjusting the planned rod contour based on the spinal contour of the patient to generate a modified contour.

11. The method of claim 10, further comprising bending a spinal rod according to the modified contour.

12. The method of claim 10, wherein the flexible surgical sensor is received by one or more pedicle screws to facilitate conformity of the flexible surgical sensor with the spinal contour.

13. A system for facilitating spinal surgery comprising:
a non-transitory computer-readable storage medium configured to store a planned rod contour;
a flexible surgical sensor configured to measure a spinal contour of a patient; and
a display device configured to overlay the planned rod contour with the spinal contour.

14. The system of claim 13, further comprising a rod bender configured to bend a spinal rod according to the planned rod contour.

15. The system of claim 13, wherein the spinal contour of the patient is a current spinal contour of the patient.

16. The system of claim 13, wherein the spinal contour of the patient is a desired spinal contour of the patient.

17. The system of claim 13, wherein the non-transitory computer-readable storage medium is further configured to store data from the flexible surgical sensor.

18. The system of claim 13, wherein the flexible surgical sensor is configured to be received by one or more pedicle screws to facilitate conformity of the flexible surgical sensor with the spinal contour.

19. The system of claim 13, further comprising a processor configured to receive adjustment parameters from a user, and based on the adjustment parameters, generate a modified contour.

20. The system of claim 13, further comprising a processor configured to receive user modifications to the planned rod contour, and to modify the planned rod contour per the user modifications to generate a modified contour.
